(19) United States
(12) Patent Application Publication
(10) Pub. No.: US 2025/0304866 A1
(43) Pub. Date: Oct. 2, 2025

(54) LIQUID FUEL MANUFACTURING METHOD

(71) Applicant: HONDA MOTOR CO., LTD., Tokyo (JP)

(72) Inventors: Kota Okamura, Wako-shi (JP); Shiori Nakajima, Wako-shi (JP); Akira Sonoda, Wako-shi (JP)

(21) Appl. No.: 19/087,651

(22) Filed: Mar. 24, 2025

(30) Foreign Application Priority Data
Mar. 29, 2024 (JP) 2024-055471

(51) Int. Cl. C10J 3/72

(52) U.S. Cl. CPC C10J 3/723; C10J 2300/0923; C10J 2300/1659; C10J 2300/1684

(57) ABSTRACT

What is provided is a liquid fuel manufacturing method in which efficiency can be achieved throughout the entire system. A liquid fuel manufacturing method has a hydrogen stock quantity checking step of checking a hydrogen stock quantity, a gasifying step of producing synthesis gas from a biomass raw material, an electrolyzing step of producing hydrogen from water by means of electricity of renewable energy, and a liquid fuel manufacturing step of manufacturing liquid fuel with synthesis gas produced in the gasifying step and hydrogen produced in the electrolyzing step as raw materials. In the liquid fuel manufacturing step, the amount of supplied water vapor is decreased stepwise when an H₂/CO ratio is not smaller than a target lower limit value, and then the hydrogen stock quantity checking step is carried out when the H₂/CO ratio becomes equal to or smaller than the target lower limit value. The amount of supplied water vapor is reverted to an immediately preceding amount when there is no hydrogen stock. The amount of supplied water vapor is decreased stepwise until the amount of produced carbon monoxide stops increasing when there is hydrogen stock, and the amount of supplied water vapor is reverted to the immediately preceding amount, hydrogen is supplied such that the H₂/CO ratio becomes equivalent to the lower limit for the target value, and the hydrogen stock quantity checking step is carried out when carbon monoxide stops increasing.

### TECHNICAL FIELD

The present invention relates to a liquid fuel manufacturing method.

### BACKGROUND ART

In recent years, electrosynthetic fuel made from raw materials such as hydrogen produced by means of electricity generated using renewable energy and carbon sources such as biomass and carbon dioxide emitted from factories, has been attracting attention as a substitute for fossil fuels.

A general procedure of manufacturing liquid fuel such as methanol or gasoline using biomass as a raw material is as follows. That is, a liquid fuel is manufactured from biomass raw materials through a gasifying step of gasifying biomass raw materials that have undergone a predetermined pretreatment together with hydrogen, oxygen, and water vapor inside a gasification furnace and producing synthesis gas containing hydrogen and carbon monoxide, a cleaning step of cleaning the produced synthesis gas and removing tar, an H₂/CO ratio adjusting step of adjusting the H₂/CO ratio of the synthesis gas that has undergone the cleaning step to a target ratio corresponding to a liquid fuel intended to be manufactured, a desulfurizing step of removing sulfur components from the synthesis gas that has undergone the H₂/CO ratio adjusting step, and a fuel manufacturing step of manufacturing liquid fuel from the synthesis gas that has undergone the desulfurizing step.

### CITATION LIST

### Patent Document

Patent Document 1: Japanese Unexamined Patent Application, First Publication No. 2021-147504

### SUMMARY OF INVENTION

### Technical Problem

In a gasifying step, biomass is pyrolyzed and mixed gas containing hydrogen, carbon monoxide, carbon dioxide, methane, and the like is generated. Among gases generated due to pyrolysis, hydrogen and carbon monoxide become raw materials for liquid fuel. When a liquid fuel is made from hydrogen and carbon monoxide, there is a need to control the index such as an H₂/CO ratio to be within an appropriate value. While the H₂/CO ratio is controlled to be within an appropriate range, if a gasification furnace can be operated on condition that the amount of carbon monoxide generated is maximized within the range, the yield rate of liquid fuel can be maximized.

The present invention has been made in consideration of the foregoing problems, and an object thereof is to provide a liquid fuel manufacturing method in which efficiency can be achieved throughout the entire system and which will contribute to improvement in quality control in manufacturing steps and ultimately to energy efficiency.

### Solution to Problem

In order to achieve the foregoing object, the present invention provides the following means.
[1] There is provided a liquid fuel manufacturing method for manufacturing liquid fuel from a biomass raw material. The liquid fuel manufacturing method has a hydrogen stock quantity checking step of checking a hydrogen stock quantity, a gasifying step of producing synthesis gas from a biomass raw material, an electrolyzing step of producing hydrogen from water by means of electricity generated using renewable energy, and a liquid fuel manufacturing step of manufacturing liquid fuel with synthesis gas produced through the gasifying step and hydrogen produced through the electrolyzing step as raw materials. In the liquid fuel manufacturing step, the amount of supplied water vapor is decreased stepwise when an H₂/CO ratio is not smaller than a target lower limit value, and then the hydrogen stock quantity checking step is carried out when the H₂/CO ratio becomes equal to or smaller than the target lower limit value. The amount of supplied water vapor is reverted to an immediately preceding amount when there is no hydrogen stock. The amount of supplied water vapor is decreased stepwise until the amount of produced carbon monoxide stops increasing when there is hydrogen stock, and the amount of supplied water vapor is reverted to the immediately preceding amount, hydrogen is supplied such that the H₂/CO ratio becomes equivalent to the lower limit for the target value, and the hydrogen stock quantity checking step is carried out when carbon monoxide stops increasing.

The present invention can achieve a target range of the H₂/CO ratio and maximization of the amount of carbon monoxide regardless of the presence or absence of hydrogen, operating conditions of a gasification furnace, and the kind of biomass used.
[2] In the liquid fuel manufacturing method according to [1], in the liquid fuel manufacturing step, when there is no hydrogen stock, the amount of supplied water vapor is adjusted to become intermediate between the initial amount of supplied water vapor and the current amount of supplied water vapor, hydrogen is supplied such that the H₂/CO ratio becomes equivalent to the target lower limit value, and the amount of supplied water vapor is repeatedly adjusted until hydrogen is in stock.

The present invention can achieve a target range of the H₂/CO ratio and maximization of the amount of carbon monoxide regardless of the presence or absence of hydrogen, operating conditions of a gasification furnace, and the kind of biomass used.
[3] There is provided a liquid fuel manufacturing method for manufacturing liquid fuel from a biomass raw material. The liquid fuel manufacturing method has a hydrogen stock quantity checking step of checking a hydrogen stock quantity, a gasifying step of producing synthesis gas from a biomass raw material, an electrolyzing step of producing hydrogen from water by means of electricity generated using renewable energy, and a liquid fuel manufacturing step of manufacturing liquid fuel with synthesis gas produced through the gasifying step and hydrogen produced through the electrolyzing step as raw materials. In the liquid fuel manufacturing step, the amount of supplied water vapor is increased stepwise when an H₂/CO ratio is smaller than a target lower limit value, the amount of supplied water vapor is repeatedly increased stepwise until the amount of produced carbon monoxide stops increasing when the amount of produced carbon monoxide has increased, the amount of supplied water vapor is decreased stepwise when the amount of produced carbon monoxide is not increasing, and the amount of supplied water vapor is reverted to an immediately preceding amount, hydrogen is supplied such that the H₂/CO ratio becomes equivalent to the target lower limit value, and the hydrogen stock quantity checking step is carried out when the amount of produced carbon monoxide is not increasing.

The present invention can achieve a target range of the H₂/CO ratio and maximization of the amount of carbon monoxide regardless of the presence or absence of hydrogen, operating conditions of a gasification furnace, and the kind of biomass used.
[4] In the liquid fuel manufacturing method according to [3], in the liquid fuel manufacturing step, when there is no hydrogen stock, the amount of supplied water vapor is adjusted to become intermediate between an initial amount of supplied water vapor and a current amount of supplied water vapor, hydrogen is supplied such that the H₂/CO ratio becomes equivalent to the target lower limit value, and the amount of supplied water vapor is repeatedly adjusted until the amount of supplied hydrogen becomes smaller than the amount of produced hydrogen.

The present invention can achieve a target range of the H₂/CO ratio and maximization of the amount of carbon monoxide regardless of the presence or absence of hydrogen, operating conditions of a gasification furnace, and the kind of biomass used.
[5] In the liquid fuel manufacturing method according to [3], in the liquid fuel manufacturing step, the amount of supplied water vapor is decreased stepwise when the amount of produced carbon monoxide is not increasing, hydrogen is supplied such that the H₂/CO ratio becomes equivalent to the target lower limit value when the amount of produced carbon monoxide has increased, and the amount of supplied water vapor is reverted to the immediately preceding amount and hydrogen is supplied such that the H₂/CO ratio becomes equivalent to the target lower limit value when hydrogen is not in stock.

The present invention can achieve a target range of the H₂/CO ratio and maximization of the amount of carbon monoxide regardless of the presence or absence of hydrogen, operating conditions of a gasification furnace, and the kind of biomass used.

### Advantageous Effects of Invention

According to the present invention, it is possible to provide a liquid fuel manufacturing method in which efficiency can be achieved throughout the entire system.

### BRIEF DESCRIPTION OF DRAWINGS

FIG. 1 A view showing the constitution of a fuel manufacturing system used in a liquid fuel manufacturing method according to an embodiment of the present invention.

FIG. 2 A flowchart showing the liquid fuel manufacturing method according to the embodiment of the present invention.

FIG. 3 A flowchart showing the liquid fuel manufacturing method according to the embodiment of the present invention.

### DESCRIPTION OF EMBODIMENTS

Hereinafter, a liquid fuel manufacturing method according to an embodiment of the present invention will be described with reference to the drawings.

### [Fuel Manufacturing System]

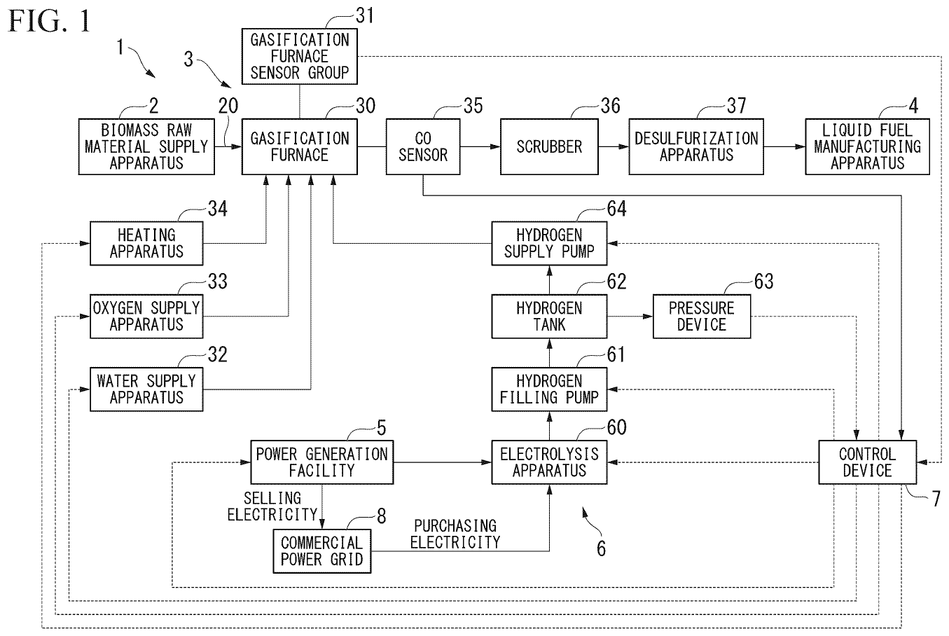

FIG. 1 is a view showing the constitution of a fuel manufacturing system used in the liquid fuel manufacturing method according to the embodiment of the present invention.

As shown in FIG. 1, a fuel manufacturing system 1 includes a biomass raw material supply apparatus 2 supplying a biomass raw material, a gasification apparatus 3 gasifying a biomass raw material supplied from the biomass raw material supply apparatus 2 and producing synthesis gas containing hydrogen and carbon monoxide, a liquid fuel manufacturing apparatus 4 manufacturing liquid fuel with synthesis gas supplied from the gasification apparatus 3 and hydrogen produced by an electrolysis apparatus 60 as raw materials, a power generation facility 5 generating electricity using renewable energy, a hydrogen production supply apparatus 6 producing hydrogen and oxygen from water by means of electricity generated by the power generation facility 5 and supplying produced hydrogen and oxygen to the gasification apparatus 3, and a control device 7 controlling the gasification apparatus 3, the power generation facility 5, and the hydrogen production supply apparatus 6, thereby manufacturing liquid fuel from a biomass raw material using these.

The biomass raw material supply apparatus 2 performs a predetermined pretreatment with respect to a biomass raw material such as rice hulls, bagasse, or wood and supplies the biomass raw material that has undergone this pretreatment to a gasification furnace 30 of the gasification apparatus 3 via a raw material supply path 20. Here, for example, the pretreatment with respect to a biomass raw material includes a drying step of drying raw materials, a crushing step of crushing raw material, and the like.

The gasification apparatus 3 includes the gasification furnace 30 gasifying a biomass raw material supplied via the raw material supply path 20, a gasification furnace sensor group 31 constituted of a plurality of sensors determining the state inside the gasification furnace 30, a water supply apparatus 32 supplying water to the inside of the gasification furnace 30, an oxygen supply apparatus 33 supplying oxygen or air to the inside of the gasification furnace 30, a heating apparatus 34 heating the gasification furnace 30, a CO sensor 35 measuring the concentration of carbon monoxide contained in synthesis gas emitted from the gasification furnace 30, a scrubber 36 cleaning synthesis gas emitted from the gasification furnace 30, and a desulfurization apparatus 37 eliminating sulfur components from synthesis gas cleaned by the scrubber 36 and supplying a result to the liquid fuel manufacturing apparatus 4.

The water supply apparatus 32 supplies water retained in a water tank (not shown) to the inside of the gasification furnace 30. The oxygen supply apparatus 33 supplies oxygen retained in an oxygen tank (not shown) to the inside of the gasification furnace 30. The heating apparatus 34 heats the gasification furnace 30 by consuming fuel supplied from a fuel tank (not shown) or electricity supplied from a power source (not shown). The amount of supplied water from the water supply apparatus 32 to the inside of the gasification furnace 30, the amount of supplied oxygen from the oxygen supply apparatus 33 to the inside of the gasification furnace 30, and the amount of input heat from the heating apparatus 34 to the gasification furnace 30 are controlled by the control device 7. In the fuel manufacturing system 1, there may be no need to actively supply water from the water supply apparatus 32 to the inside of the gasification furnace 30 by supplying hydrogen from the hydrogen production supply apparatus 6 (which will be described below) to the inside of the gasification furnace 30 or to the inside of the raw material supply path 20. In this case, the water supply apparatus 32 can be excluded from the fuel manufacturing system 1.

If water, oxygen, heat, and the like are input to the inside of the gasification furnace 30, to which a biomass raw material has been input, by the water supply apparatus 32, the oxygen supply apparatus 33, and the heating apparatus 34, for example, ten kinds of gasification reactions and reverse reactions thereof in total shown in the following formulas (1-1) to (1-5) progress, and synthesis gas containing hydrogen and carbon monoxide is produced inside the gasification furnace 30.

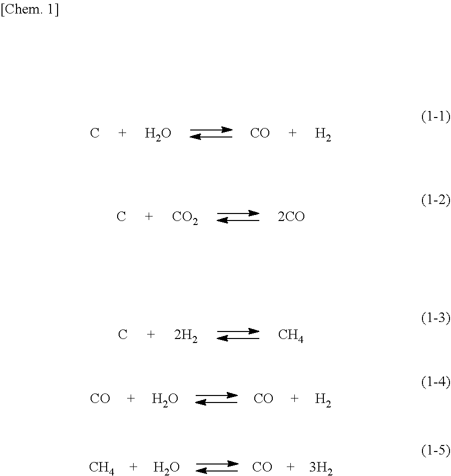

For example, the gasification furnace sensor group 31 is constituted of a pressure sensor for determining the pressure inside the gasification furnace 30, a temperature sensor for determining the temperature inside the gasification furnace 30, an H₂/CO sensor for determining the H₂/CO ratio corresponding to the ratio of hydrogen to carbon monoxide of synthesis gas inside the gasification furnace 30, a CO₂ sensor for determining carbon dioxide inside the gasification furnace 30, and the like. Determination signals of these sensors constituting the gasification furnace sensor group 31 are transmitted to the control device 7.

The gasification apparatus 3 adjusts the H₂/CO ratio of synthesis gas to a predetermined target ratio (for example, when methanol is manufactured, the target ratio of the H₂/CO ratio is 2) corresponding to the liquid fuel intended to be manufactured by mixing hydrogen supplied from the hydrogen production supply apparatus 6 (which will be described below) with synthesis gas produced due to the gasification reaction and reverse reaction thereof shown in the foregoing formulas (1-1) to (1-5), and then supplies this synthesis gas to the liquid fuel manufacturing apparatus 4.

The liquid fuel manufacturing apparatus 4 includes a methanol synthesis apparatus, a methanol-to-gasoline (MTG) synthesis apparatus, a Fischer-Tropsch (FT) synthesis apparatus, an upgrading device, and the like and manufactures liquid fuel such as methanol or gasoline from synthesis gas adjusted to a predetermined H₂/CO ratio in the gasification apparatus 3 using these.

The power generation facility 5 is constituted of a wind power generation facility generating electricity using wind power that is renewable energy, a solar power generation facility generating electricity using sunlight that is renewable energy, or the like. The power generation facility 5 is connected to the hydrogen production supply apparatus 6, and electricity generated using renewable energy in the wind power generation facility, a solar power generation facility, or the like can be supplied to the hydrogen production supply apparatus 6. In addition, the power generation facility 5 is also connected to a commercial power grid 8. For this reason, a part or all of electricity generated in the power generation facility 5 can also be sold to a power company by being supplied to the commercial power grid 8.

The hydrogen production supply apparatus 6 includes the electrolysis apparatus 60, a hydrogen filling pump 61, a hydrogen tank 62, a pressure sensor 63, and a hydrogen supply pump 64, uses these to produce hydrogen by means of electricity supplied from the power generation facility 5, and supplies produced hydrogen to the gasification apparatus 3.

The electrolysis apparatus 60 is connected to the power generation facility 5 and produces hydrogen and oxygen from water through electrolysis by means of electricity supplied from the power generation facility 5. In addition, the electrolysis apparatus 60 is also connected to the commercial power grid 8. For this reason, the electrolysis apparatus 60 can produce hydrogen and oxygen not only by means of electricity supplied from the power generation facility 5 but also by means of electricity supplied from the commercial power grid 8 by purchasing electricity from a power company. The amount of hydrogen and the amount of oxygen produced by the electrolysis apparatus 60 are controlled by the control device 7.

The hydrogen filling pump 61 compresses hydrogen produced by the electrolysis apparatus 60 and fills the inside of the hydrogen tank 62. The amount of hydrogen filled by the hydrogen filling pump 61 is controlled by the control device 7. The hydrogen tank 62 retains hydrogen compressed by the hydrogen filling pump 61. The pressure sensor 63 determinates the tank internal pressure of the hydrogen tank 62 and transmits determination signals to the control device 7. The amount of hydrogen remaining inside the hydrogen tank 62 is calculated by the control device 7 on the basis of determination signals of the pressure sensor 63. Therefore, in the present embodiment, a hydrogen remaining amount acquisition means for acquiring the amount of hydrogen remaining inside the hydrogen tank 62 is constituted of the pressure sensor 63 and the control device 7.

The hydrogen supply pump 64 supplies hydrogen retained in the hydrogen tank 62 to the inside of the gasification furnace 30 of the gasification apparatus 3. The amount of supplied hydrogen from the hydrogen supply pump 64 to the inside of the gasification furnace 30 is controlled by the control device 7. In FIG. 1, a case in which hydrogen retained in the hydrogen tank 62 is supplied to the inside of the gasification furnace 30 by the hydrogen supply pump 64 will be described, but the present invention is not limited thereto. Hydrogen retained in the hydrogen tank 62 may be supplied to the upstream side of the gasification furnace 30, more specifically to the inside of the raw material supply path 20 for a biomass raw material.

The control device 7 is a computer controlling the amount of water supplied by the water supply apparatus 32, the amount of oxygen supplied by the oxygen supply apparatus 33, the amount of heat input by the heating apparatus 34, the amount of hydrogen produced by the electrolysis apparatus 60, the amount of hydrogen filled by the hydrogen filling pump 61, and the amount of hydrogen supplied by the hydrogen supply pump 64 on the basis of determination signals from the gasification furnace sensor group 31, determination signals from the pressure sensor 63 of the hydrogen tank 62, and the like.

### Liquid Fuel Manufacturing Method

### First Embodiment

A liquid fuel manufacturing method according to the embodiment of the present invention is a liquid fuel manufacturing method for manufacturing liquid fuel from a biomass raw material. The method has a hydrogen stock quantity checking step of checking a hydrogen stock quantity, a gasifying step of producing synthesis gas from a biomass raw material, an electrolyzing step of producing hydrogen from water by means of electricity generated using renewable energy, and a liquid fuel manufacturing step of manufacturing liquid fuel with synthesis gas produced through the gasifying step and hydrogen produced through the electrolyzing step as raw materials. In the liquid fuel manufacturing step, the amount of supplied water vapor is decreased stepwise when an H₂/CO ratio is not smaller than a target lower limit value, and then the hydrogen stock quantity checking step is carried out when the H₂/CO ratio becomes equal to or smaller than the target lower limit value. The amount of supplied water vapor is reverted to an immediately preceding amount when there is no hydrogen stock. The amount of supplied water vapor is decreased stepwise until the amount of produced carbon monoxide stops increasing when there is hydrogen stock, and the amount of supplied water vapor is reverted to the immediately preceding amount, hydrogen is supplied such that the H₂/CO ratio becomes equivalent to the lower limit for the target value, and the hydrogen stock quantity checking step is carried out when carbon monoxide stops increasing.

In the present specification, the expression “immediately preceding amount” denotes a supply amount in an immediately preceding step in a stepwise decrease or increase of supply amount.

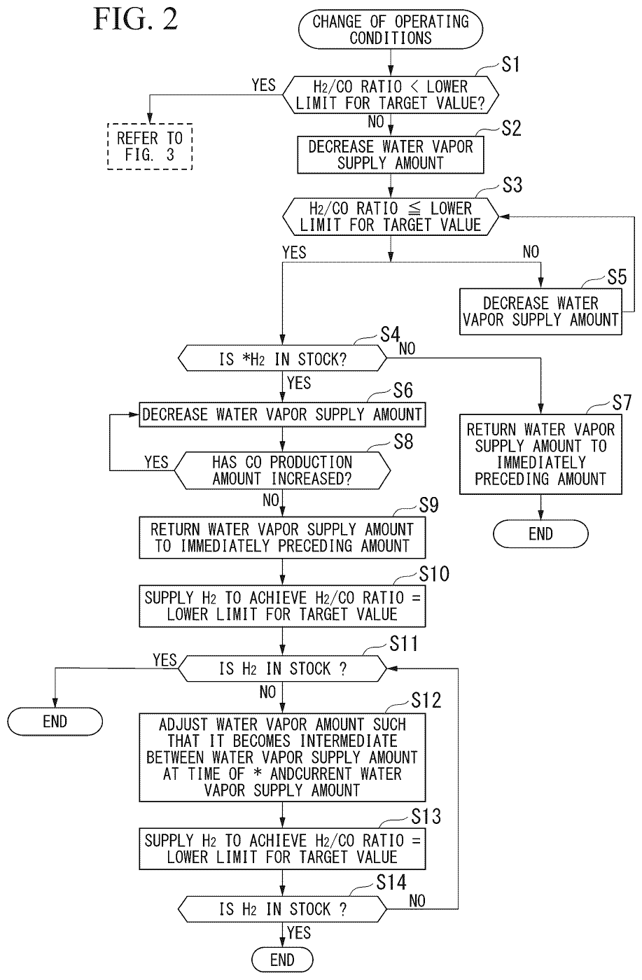

With reference to FIG. 2, the liquid fuel manufacturing method of the present embodiment will be described.

FIG. 2 is a flowchart showing a specific procedure of the liquid fuel manufacturing method of the present embodiment. It is checked whether or not the H₂/CO ratio of synthesis gas generated by the gasification furnace 30 is smaller than the target lower limit value (Step S1).

When the H₂/CO ratio is not smaller than the target lower limit value (when NO), the amount of water vapor supplied to the gasification apparatus 3 is decreased stepwise (Step S2).

Thereafter, it is checked whether or not the H₂/CO ratio has become equal to or smaller than the target lower limit value (Step S3). When the H₂/CO ratio has become equal to or smaller than the target lower limit value (when YES), the hydrogen stock quantity checking step is carried out (Step S4).

When the H₂/CO ratio exceeds the target lower limit value (when NO), the amount of water vapor supplied to the gasification apparatus 3 is decreased stepwise (Step S5), and the processing returns to Step S3 again.

In Step S4, when there is hydrogen stock (when YES), the amount of water vapor supplied to the gasification apparatus 3 is decreased stepwise (Step S6).

In Step S4, when there is no hydrogen stock (when NO), the amount of supplied water vapor is reverted to the immediately preceding amount (Step S7).

In Step S6, the amount of water vapor supplied to the gasification apparatus 3 is decreased stepwise, and then it is determined whether or not the amount of produced carbon monoxide has increased (Step S8). When the amount of produced carbon monoxide has increased (when YES), the processing returns to Step S6, and the amount of water vapor supplied to the gasification apparatus 3 is decreased stepwise. When the amount of produced carbon monoxide has decreased (when NO in the state in which the amount of produced carbon monoxide has stopped increasing), the amount of supplied water vapor is reverted to the immediately preceding amount (Step S9).

Thereafter, hydrogen is supplied to the gasification apparatus 3 such that the H₂/CO ratio becomes equivalent to the target lower limit value (Step S10).

Thereafter, the hydrogen stock quantity checking step is carried out (Step S11).

In Step S11, when there is hydrogen stock (when YES), the current amount of hydrogen supplied to the gasification apparatus 3 is maintained.

In Step S11, when there is no hydrogen stock (when NO), the amount of water vapor supplied to the gasification apparatus 3 is adjusted to become intermediate between the initial amount of supplied water vapor and the current amount of supplied water vapor (Step S12).

Thereafter, hydrogen is supplied to the gasification apparatus 3 such that the H₂/CO ratio becomes equivalent to the target lower limit value (Step S13).

Thereafter, the hydrogen stock quantity checking step is carried out (Step S14). In Step S14, when there is hydrogen stock (when YES), the current amount of hydrogen supplied to the gasification apparatus 3 is maintained. In Step S14, when there is no hydrogen stock (when NO), the amount of supplied water vapor is repeatedly adjusted until hydrogen is in stock.

According to the liquid fuel manufacturing method of the present embodiment, it is possible to achieve a target range of the H₂/CO ratio and maximization of the amount of carbon monoxide regardless of the presence or absence of hydrogen, operating conditions of a gasification furnace, and the kind of biomass used. Therefore, it is possible to achieve efficiency throughout the entire liquid fuel manufacturing system.

### Second Embodiment

A liquid fuel manufacturing method according to another embodiment of the present invention is a liquid fuel manufacturing method for manufacturing liquid fuel from a biomass raw material. The method has a gasifying step of producing synthesis gas from a biomass raw material, an electrolyzing step of producing hydrogen from water by means of electricity generated using renewable energy, and a liquid fuel manufacturing step of manufacturing liquid fuel with synthesis gas produced through the gasifying step and hydrogen produced through the electrolyzing step as raw materials. In the liquid fuel manufacturing step, the amount of supplied water vapor is increased stepwise when an H₂/CO ratio is smaller than a target lower limit value, the amount of supplied water vapor is repeatedly increased until the amount of produced carbon monoxide stops increasing when the amount of produced carbon monoxide has increased, the amount of supplied water vapor is decreased stepwise when the amount of produced carbon monoxide is not increasing, and the amount of supplied water vapor is reverted to the immediately preceding amount, hydrogen is supplied such that the H₂/CO ratio becomes equivalent to the target lower limit value, and the hydrogen stock quantity is checked when the amount of produced carbon monoxide is not increasing.

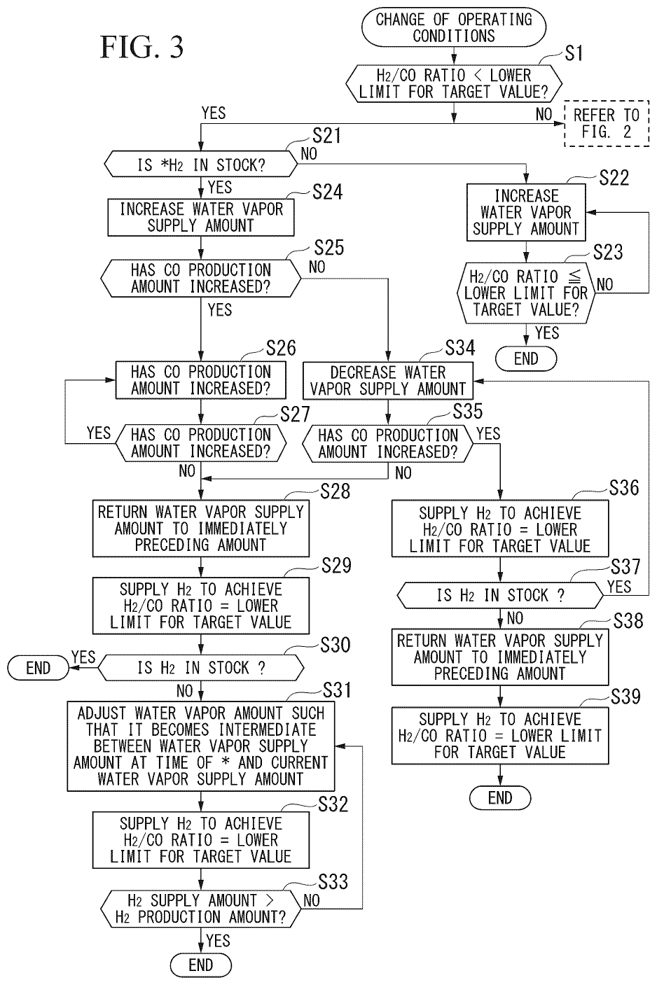

With reference to FIG. 3, the liquid fuel manufacturing method of the present embodiment will be described.

FIG. 3 is a flowchart showing a specific procedure of the liquid fuel manufacturing method of the present embodiment. It is checked whether or not the H₂/CO ratio of synthesis gas generated by the gasification furnace 30 is smaller than the target lower limit value (Step S1).

When the H₂/CO ratio is smaller than the target lower limit value (when YES), the hydrogen stock quantity checking step is carried out (Step S21).

In Step S21, when there is no hydrogen stock (when NO), the amount of water vapor supplied to the gasification apparatus 3 is increased stepwise (Step S22).

Thereafter, it is checked whether or not the H₂/CO ratio has become equal to or smaller than the target lower limit value (Step S23). In Step S23, when the H₂/CO ratio has become equal to or smaller than the target lower limit value, the current amount of water vapor supplied to the gasification apparatus 3 is maintained. In Step S23, when the H₂/CO ratio is not equal to or smaller than the target lower limit value, the processing returns to Step S22, and the amount of water vapor supplied to the gasification apparatus 3 is increased stepwise.

In Step S21, when there is hydrogen stock (when YES), the amount of water vapor supplied to the gasification apparatus 3 is increased stepwise (Step S24).

Thereafter, it is checked whether or not the amount of produced carbon monoxide has increased (Step S25).

In Step S25, when the amount of produced carbon monoxide has increased (when YES), the amount of water vapor supplied to the gasification apparatus 3 is increased stepwise (Step S26).

Thereafter, it is checked whether or not the amount of produced carbon monoxide has increased (Step S27).

In Step S27, when the amount of produced carbon monoxide has increased (when YES), the processing returns to Step S26, and the amount of water vapor supplied to the gasification apparatus 3 is increased stepwise.

In Step S27, when the amount of produced carbon monoxide is not increasing (when NO), the amount of supplied water vapor is reverted to the immediately preceding amount (Step S28).

Thereafter, hydrogen is supplied to the gasification apparatus 3 such that the H₂/CO ratio becomes equivalent to the target lower limit value (Step S29).

Thereafter, the hydrogen stock quantity checking step is carried out (Step S30). In Step S30, when there is hydrogen stock (when YES), the current amount of hydrogen supplied to the gasification apparatus 3 is maintained. In Step S30, when there is no hydrogen stock (when NO), the amount of water vapor supplied to the gasification apparatus 3 is adjusted to become intermediate between the initial amount of supplied water vapor and the current amount of supplied water vapor (Step S31).

Thereafter, hydrogen is supplied to the gasification apparatus 3 such that the H₂/CO ratio becomes equivalent to the target lower limit value (Step S32).

Thereafter, the hydrogen stock quantity checking step is carried out (Step S33). In Step S33, when there is hydrogen stock (when YES), the current amount of hydrogen supplied to the gasification apparatus 3 is maintained. In Step S33, when there is no hydrogen stock (when NO), the amount of supplied water vapor is repeatedly adjusted until hydrogen is in stock.

In Step S25, when the amount of produced carbon monoxide is not increasing (when NO), the amount of water vapor supplied to the gasification apparatus 3 is decreased stepwise (Step S34).

Thereafter, it is checked whether or not the amount of produced carbon monoxide has increased (Step S35).

In Step S35, when the amount of produced carbon monoxide is not increasing (when NO), the processing shifts to Step S28.

In Step S35, when the amount of produced carbon monoxide has increased (when YES), hydrogen is supplied to the gasification apparatus 3 such that the H₂/CO ratio becomes equivalent to the target lower limit value (Step S36).

Thereafter, the hydrogen stock quantity checking step is carried out (Step S37). In Step S37, when there is hydrogen stock (when YES), the processing returns to Step S34, and the amount of water vapor supplied to the gasification apparatus 3 is decreased stepwise. In Step S37, when there is no hydrogen stock (when NO), the amount of supplied water vapor is reverted to the immediately preceding amount (Step S38).

Thereafter, hydrogen is supplied to the gasification apparatus 3 such that the H₂/CO ratio becomes equivalent to the target lower limit value (Step S39).

According to the liquid fuel manufacturing method of the present embodiment, it is possible to achieve a target range of the H₂/CO ratio and maximization of the amount of carbon monoxide regardless of the presence or absence of hydrogen, operating conditions of a gasification furnace, and the kind of biomass used. Therefore, it is possible to achieve efficiency throughout the entire liquid fuel manufacturing system.

Hereinabove, embodiments of the present invention have been described in detail. However, the present invention is not limited to the foregoing embodiments, and various modifications and changes can be made within the scope of the gist of the present invention described in the claims.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

### REFERENCE SIGNS LIST

1 Fuel manufacturing system
2 Biomass raw material supply apparatus
20 Raw material supply path
3 Gasification apparatus
30 Gasification furnace
31 Gasification furnace sensor group
32 Water supply apparatus
33 Oxygen supply apparatus
34 Heating apparatus
35 CO sensor
36 Scrubber
37 Desulfurization apparatus
4 Liquid fuel manufacturing apparatus
5 Power generation facility
6 Hydrogen production supply apparatus
60 Electrolysis apparatus
61 Hydrogen filling pump
62 Hydrogen tank
63 Pressure sensor
64 Hydrogen supply pump
7 Control device

What is claimed is:

1. A liquid fuel manufacturing method for manufacturing liquid fuel from a biomass raw material, the method comprising:
a hydrogen stock quantity checking step of checking a hydrogen stock quantity;
a gasifying step of producing synthesis gas from a biomass raw material;
an electrolyzing step of producing hydrogen from water by means of electricity generated using renewable energy; and
a liquid fuel manufacturing step of manufacturing liquid fuel with synthesis gas produced through the gasifying step and hydrogen produced through the electrolyzing step as raw materials,
wherein in the liquid fuel manufacturing step, an amount of supplied water vapor is decreased stepwise when an H₂/CO ratio is not smaller than a target lower limit value, and then the hydrogen stock quantity checking step is carried out when the H₂/CO ratio becomes equal to or smaller than the target lower limit value,
the amount of supplied water vapor is reverted to an immediately preceding amount when there is no hydrogen stock, and
the amount of supplied water vapor is decreased stepwise until an amount of produced carbon monoxide stops increasing when there is hydrogen stock, and the amount of supplied water vapor is reverted to the immediately preceding amount, hydrogen is supplied such that the H₂/CO ratio becomes equivalent to the lower limit for the target value, and the hydrogen stock quantity checking step is carried out when carbon monoxide stops increasing.

2. The liquid fuel manufacturing method according to claim 1,
wherein in the liquid fuel manufacturing step, when there is no hydrogen stock, the amount of supplied water vapor is adjusted to become intermediate between an initial amount of supplied water vapor and a current amount of supplied water vapor, hydrogen is supplied such that the H₂/CO ratio becomes equivalent to the target lower limit value, and the amount of supplied water vapor is repeatedly adjusted until hydrogen is in stock.

3. A liquid fuel manufacturing method for manufacturing liquid fuel from a biomass raw material, the method comprising:
a hydrogen stock quantity checking step of checking a hydrogen stock quantity;
a gasifying step of producing synthesis gas from a biomass raw material;
an electrolyzing step of producing hydrogen from water by means of electricity generated using renewable energy; and
a liquid fuel manufacturing step of manufacturing liquid fuel with synthesis gas produced through the gasifying step and hydrogen produced through the electrolyzing step as raw materials,
wherein in the liquid fuel manufacturing step, an amount of supplied water vapor is increased stepwise when an H₂/CO ratio is smaller than a target lower limit value, the amount of supplied water vapor is repeatedly increased stepwise until an amount of produced carbon monoxide stops increasing when the amount of produced carbon monoxide has increased, the amount of supplied water vapor is decreased stepwise when the amount of produced carbon monoxide is not increasing, and the amount of supplied water vapor is reverted to an immediately preceding amount, hydrogen is supplied such that the H₂/CO ratio becomes equivalent to the target lower limit value, and the hydrogen stock quantity checking step is carried out when the amount of produced carbon monoxide is not increasing.

4. The liquid fuel manufacturing method according to claim 3,
wherein in the liquid fuel manufacturing step, when there is no hydrogen stock, the amount of supplied water vapor is adjusted to become intermediate between an initial amount of supplied water vapor and a current amount of supplied water vapor, hydrogen is supplied such that the H₂/CO ratio becomes equivalent to the target lower limit value, and the amount of supplied water vapor is repeatedly adjusted until an amount of supplied hydrogen becomes smaller than an amount of produced hydrogen.

5. The liquid fuel manufacturing method according to claim 3,
wherein in the liquid fuel manufacturing step, the amount of supplied water vapor is decreased stepwise when the amount of produced carbon monoxide is not increasing, hydrogen is supplied such that the H₂/CO ratio becomes equivalent to the target lower limit value when the amount of produced carbon monoxide has increased, and the amount of supplied water vapor is reverted to the immediately preceding amount and hydrogen is supplied such that the H₂/CO ratio becomes equivalent to the target lower limit value when hydrogen is not in stock.
